(19) United States
(12) Patent Application Publication
(10) Pub. No.: US 2017/0238848 A1
(43) Pub. Date: Aug. 24, 2017

(54) Device, System & Method for Improving Fitness Posture

(71) Applicant: Dayna Goldstein, Statesboro, GA (US)

(72) Inventor: Dayna Goldstein, Statesboro, GA (US)

(21) Appl. No.: 15/052,092

(22) Filed: Feb. 24, 2016

(51) Int. Cl. A61B 5/11; A63B 24/00; G09B 5/02

(52) U.S. Cl. CPC A61B 5/1116; G09B 5/02; A63B 24/0062; A63B 24/0003

(57) ABSTRACT

Certain embodiments herein relate to a device, system, and method for improving fitness posture. In one embodiment, a system for improving fitness posture can be provided. The system can include at least one memory to store computer-executable instructions and a processor configured to access the at least one memory and execute the computer-executable instructions. The instructions may be configured to receive, from one or more sensors, exercise information associated with the movement of a user while performing an exercise. The instruction can be further configured to determine if the user is performing the exercise correctly based at least in part on the exercise information. The instructions can be further configured to generate one or more messages to provide feedback to the user about improving the user's posture and performance of the exercise. The instructions can be further configured to transmit the messages to a user device associated with the user.

### FIELD OF THE DISCLOSURE

Embodiments of the disclosure generally relate to a device, system and method for improving fitness posture, and more particularly, to a device with a laser that provides a body posture aid as well as a sensor-based feedback system that facilitates proper exercise technique.

### BACKGROUND

People today are involved in sedentary jobs and hence are more prone to bad body posture. As such, exercise has become an essential part of a daily routine and is important in maintaining good health. A number of studies have revealed the benefits of regular physical exercise. However, if body posture is not correctly followed while exercising, then the benefits of exercise may be negated.

Typically, a person may hire a fitness professional or trainer to facilitate proper exercising and to teach about the correct forms and techniques of when performing exercises. The presence of a trainer helps an individual to perform the exercise correctly. Additionally, expert trainers are able to detect and correct any faults occurring in the exercise performance of the individual and provide instructions about the correct manner of performing the exercise. However, often times, personal trainers are expensive and people simply cannot afford to pay for a personal fitness instructor. Furthermore, when an individual is performing a fast or complex exercise movement, it may be difficult for the trainer to accurately analyze the exercise movement of the individual.

### SUMMARY

Some or all of the above needs and/or problems may be addressed by certain embodiments of the disclosure. According to one embodiment of the disclosure, there is disclosed a device for improving fitness posture. The device for improving fitness posture may comprise a housing unit that is configured to attach to the upper body of a user. The housing unit may comprise at least one aperture. The device for improving fitness posture may further comprise a laser that is disposed within the housing unit and behind the at least one aperture, where the laser is configured to project a light through the aperture and onto a surface in front of the user in order to aid the user while performing one or more exercises. The device for improving fitness performance may further comprise one or more sensors that are disposed within the housing unit that are configured to collect exercise information associated with the movement of the user while performing one or more exercises. The device for improving fitness posture may further comprise at least one processor disposed within the housing unit that is in communication within the one or more sensors. The at least one processor is configured to receive the exercise information from the one or more sensors in order to determine if the user is the performing the one or more exercises correctly. The at least one processor is further configured to generate one or more messages to provide feedback to the user about improving the user's posture and/or performance while performing the one or more exercises and transmit the one or more messages to a user device associated with the user.

According to another embodiment of the disclosure, there is disclosed a system for improving fitness posture. The system may include one or more sensors configured to collect exercise information associated with the movement of a user while performing an exercise. The system may further include at least one memory configured to store computer-executable instructions and at least one processor configured to access the at least one memory and execute the computer-executable instructions. The instructions may be configured to receive the exercise information from the one or more sensors. The instructions may be further configured to determine whether the user is performing the exercise correctly based at least on the exercise data. The instructions may be further configured to generate one or more messages to provide feedback to the user about improving the user's posture and/or performance while performing the exercise. The instructions may be further configured to transmit the one or more messages to a user device associated with the user.

According to yet another embodiment of the disclosure, there is disclosed a method for improving fitness posture. The method may include receiving exercise information from the one or more sensors, devices, or computing devises, where the exercise information comprises one or more values or measurements associated with the movement or rotation a user while performing an exercise. The method may further include determining if the user is performing the exercise correctly based at least in part on the exercise information. The method may further include generating, based at least in part on the determination, one or more messages to provide feedback to the user about improving the user's posture and performance of the exercise. The method may further include transmitting the one or more messages to a user device associated with the user.

Accordingly, a feature and advantage of the present disclosure is its ability to assist in the development of general physical fitness.

Another feature and advantage of the present disclosure is its ability to provide a handy device that maintains body posture of a user during the performance of specific exercises.

A further feature and advantage of the present disclosure is its ability to provide a fitness device that is easy to use and cost effective.

Yet another feature and advantage of the present disclosure is its ability to provide a fitness device that has a light weight assembly.

Other embodiments, aspects, and features of the disclosure will become apparent to those skilled in the art from the following detailed description, the accompanying drawings, and the appended claims.

### BRIEF DESCRIPTION OF THE DRAWINGS

The detailed description is set forth with reference to the accompanying drawings, which are not necessarily drawn to scale. The use of the same reference numbers in different figures indicates similar or identical items.

FIG. 1 is a front view of a device for improving fitness posture, according to an embodiment of the disclosure.

FIG. 2 is a block diagram of one example system that facilitates improving fitness posture, according to an embodiment of the disclosure.

FIG. 3 is a flow diagram of an example method for improving fitness posture, according to an embodiment of the disclosure.

FIG. 4 illustrates a device for improving fitness posture being worn by a user, according to an embodiment of the disclosure.

### DETAILED DESCRIPTION

Illustrative embodiments of the disclosure will now be described more fully hereinafter with reference to the accompanying drawings, in which some, but not all embodiments of the disclosure are shown. The disclosure may be embodied in many different forms and should not be construed as limited to the embodiments set forth herein; rather, these embodiments are provided so that this disclosure will satisfy applicable legal requirements.

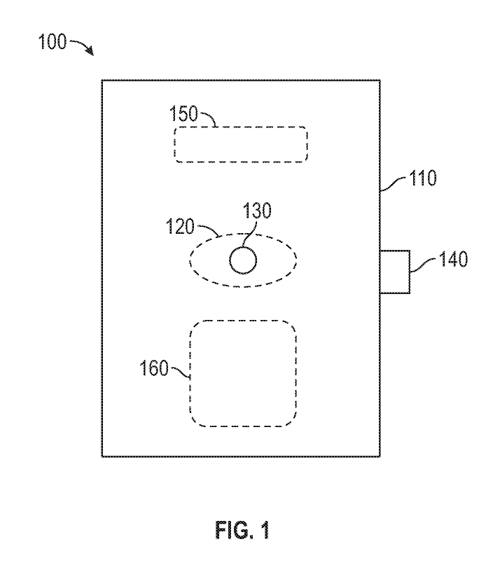

Referring now to FIG. 1, shown is a front view of a device for improving fitness posture according to an embodiments of the disclosure. As shown in FIG. 1, the device 100 may comprise housing unit 110, which may be constructed of metal, plastic, and/or any other suitable material. In certain embodiments, the device 100 is small enough to be portable and worn by a user while he/she is performing one or more exercises, such as, for example, when the user is performing squats, lifting weights, dumb-bells, straight bars, and the like. In one embodiment, the housing unit 110 of the device 100 may be approximately between 1-1.25 inches in height.

Still referring to FIG. 1, in certain embodiments, a laser 120 may be enclosed within the housing unit 110 of the device 100. The laser 120 may be any suitable laser that is capable of projecting an emission of light and may be powered by a suitable power source, such as, for instance, a battery. In certain embodiments, the housing unit 110 of the device 100 may further comprise an aperture 130, where the laser 120 may be positioned behind the aperture 130 so that light emitted by the laser 120 may escape through the aperture 130 and be projected onto, for example, a wall or an object that is positioned in front of the user in order to aid the user as he/she performs an exercises as will be described.

As shown in FIG. 1, the device 100 may further comprise one or more user controls, such as, for instance, button 140, which may be disposed on the side of the housing unit 110. In one embodiment, the button 140 may be pressed by a user in order to activate the laser 120 to emit the light. In certain embodiments, the light emitted by the laser 120 may be used as visual guidance that facilitates correct body form when the user is performing an exercise. For example, the user may attach or otherwise affix the device 100 to her or her body, such as the upper body, in order to improve their body posture when performing one or more squatting exercises. A squat is an exercise where the user may place a bar or weight on their upper back or base of the neck. While keeping their chest up, the user then performs the squat by bending their knees with their legs parallel to the floor. The user then returns to the starting positing and stands straight. In operation, when the user is in the starting position (standing straight), the user may activate the laser 120 via the button 140 so that a light may be emitted through aperture 130 and onto a wall or mirror in front of the user. As the user performs each squat, the user may continuous check to see that the light emitted by the laser 120 is projected onto the wall (not the floor) to ensure that the user keeps their head up, shoulders back, and back straight while performing one or more repetitions of the squatting exercise. Accordingly, the device 100 may facilitate proper exercise technique and posture during the performing an exercise, thereby maximizing proper fitness posture and minimizing the likelihood of injury.

With continued reference to FIG. 1, the device 100 may further comprise one or more sensors 150 and a computing device 160 that are disposed within the housing unit 110. In certain embodiments, the one or more sensors 150 may be used to monitor movements of a user's body while the user is performing an exercise. As desired, the device 100 may be positioned or mounted onto the user, where the one or more sensors 150 are configured to collect exercise information that includes information, data and/or measurements that may be used to determine whether the user is correctly performing the exercise. In certain embodiments, the one or more sensors 150 may be configured to monitor and measure three axes of linear motion of the user relative to a neutral starting position. In operation, the one or more sensors 150 may collect, at predefined intervals, exercise information, such as, values or measurements associated with the forward and backward, side-to-side, and upward and downward movement of the user's body and provide the exercise information to the computing device 160 using any number of suitable techniques.

By way of example, the device 100 may be positioned or otherwise mounted to the user's torso or upper body when the user is about to perform one or more squatting exercises. In certain embodiments, the one or more sensors 150 may collect exercise information that includes data about the user's initial starting position (i.e., when the user is in a relatively upright and neutral position). Thereafter, as the user performs the squatting exercise, the one or more sensors 150 may be configured to collect exercise information that includes measurements of the movement and rotation of the user's body relative to the starting position, such as, for example, the change in angle of the user's shoulder's and/or torso during the squat relative to the starting position (i.e., how far the user is bending over while performing the squat). Additionally, the one or more sensors 150 may be configured to collect exercise information that includes the speed and acceleration of the user while performing the squat (how fast the user is accelerating towards the floor during the squat), the number of repetitions of the squat, the distance covered during the squat (how deep the user is squatting), and the like.

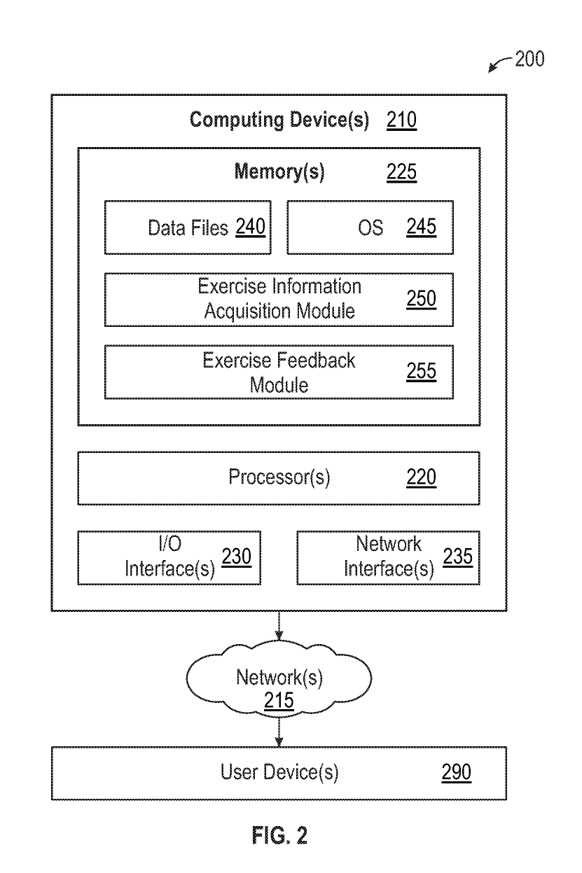

Thereafter, the one or more sensors 150 may transmit the exercise information to the computing device 160 using any number of suitable techniques. For example, the exercise information may be communicated to the computing device 160 and/or intermediary devices in communication with the computing device 160 via a suitable network, such as, such as wired, wireless, Bluetooth, and the like. In certain embodiments, the computing device 160 may include any number of suitable processing components that, amount other things, facilitate processing exercise information to determine if the user is performing the exercise correctly and also provide corrective feedback to the user regarding their exercise performance and posture, which may be communicated to a user device associated with the user (such as a personal computer, digital assistant, or mobile phone) as described further in FIG. 2. Accordingly, the technical effects of certain embodiments include the ability to provide a virtual coach that identifies incorrect form/technique and provides real-time feedback to the user about their fitness posture.

Referring now to FIG. 2, shown is a block diagram of one example system 200 that facilitates improving fitness posture according to an illustrative embodiment of the disclosure. The system 200 may include one or more computing devices 210 (referred to as 160 in FIG. 1). In certain embodiments, the one or more computing devices 210 may be used to determine whether a user is performing an exercise in a correct manner/form based on exercise information collected from one or more sensors (150 in FIG. 1). As discussed in FIG. 1, the one or more sensors may collect, at predefined intervals, exercise information, such as, values or measurements about the movement and rotation of the user while performing an exercise and provide the exercise information to the computing device 210 using any number of suitable methods or techniques as discussed above.

As desired, the computing device 210 may be in communication with any number of user devices 290 via one or more suitable networks 215, such as a local area network, a wide area network, a Foundation Fieldbus network, a Bluetooth-enabled network, and the like. Additionally, the computing device 210 may include any number of suitable processing components that may, among other things, facilitate the processing of exercise information to determine if the user is performing an exercise with the proper form and/or technique. Suitable processing devices that may be incorporated into the computing device 210 may include, but are not limited to, personal computers, server computers, application-specific circuits, microcomputing devices, minicomputers, other computing devices, and the like. As such, the computing device 210 may include any number of processors 220 that facilitate the execution of computer-readable instructions. By executing computer-readable instructions, the computing device 210 may include or form a special purpose computer or particular machine that facilitates the processing of exercise information to provide, for instance, feedback about whether the user is correctly or incorrectly performing one or more exercises.

In addition to one or more processor(s) 220, the computing device 210 may include one or more memory devices 225, one or more input/output (“I/O”) interfaces 230, and/or one or more communications and/or network interfaces 235. The one or more memory devices 225 or memories may include any suitable memory devices, for example, caches, read-only memory devices, random access memory devices, magnetic storage devices, etc. The one or more memory devices 225 may store the exercise information or other data, executable instructions, and/or various program modules utilized by the computing device 210, for example, data files 240, an operating system (“OS”) 245, an exercise information acquisition module 250 and/or an exercise feedback module 255. The data files 240 may include any suitable data that facilitates the operation of the computing device 210 including, but not limited to, exercise information collected and/or received from one or more sensors (150 in FIG. 1) disposed within device (100 in FIG. 1).

Still referring to FIG. 2, the OS 245 may include computer-executable instructions and/or program modules that may facilitate the execution of other software programs and/or program modules by the processors 220, such as, the exercise information acquisition module 250 and/or the exercise feedback module 255. The exercise information acquisition module 250 may be a suitable software module configured to receive, collect, extract and/or store live and/or historic exercise information collected about the user while the user performs one or more exercises. For instance, the exercise information acquisition module 270 may collect exercise information, such as, measurement data from the one or more sensors (150 in FIG. 1) to the computing device 210 at one or more predefined intervals, where the exercise information may be aggregated and stored in memory 225, for further processing as described below.

Thereafter, after predefined intervals or in response to some other trigger, the exercise feedback module 255 may be executed to evaluate the exercise information in real-time or near-real time to determine whether the user is performing an exercise with the correct form, posture and/or technique, thereby ensuring that the user's body is properly positioned. For instance, the exercise feedback module 255 may be configured to evaluate the exercise information, such as measurement values about the angle, roll, and pitch of the user's upper body and torso as they perform an exercise, such as a squatting motion. In certain embodiments, the exercise feedback module 255 may determine if a user is performing an exercise correctly by comparing the exercise information associated with the user to one or more pre-defined models that are associated with a specific range of motion that should be exhibited by the user when performing the exercise. Based at least in part on the comparison, the exercise feedback module 255 may then determine whether the user is performing an exercise correctly and/or provide feedback about how to improve their posture and/or performance while performing the exercise. In other embodiments, the exercise feedback module 255 may determine if a user is performing an exercise correctly by processing the exercise information via a variety of algorithms associated with recommended exercise techniques from expert techniques.

For instance, when a user performs a squatting exercise, the user's chest should remain upright (i.e. the user should not bend their chest toward the ground). As the user performs the squat, the one or more sensors (150 in FIG. 1) may collect exercise information that includes measurements about the angle of the user's chest as the user performs the squat, and then communicate this exercise information to the computing device 210. The computing device 210 may then be configured to execute the exercise feedback module 255 to process the exercise information. In one embodiment, the exercise feedback module 255 may evaluate the exercise information by comparing it to one or more predefined that may include training data. In certain embodiments, the training data may include values specifying a proper range of angles a person's chest should be within while performing a squatting exercise (and/or measurements associated with the posture of a person correctly performing the exercise). Accordingly, based at least upon the comparison, the exercise feedback module 255 may then determine if the angle of the user's chest is outside the appropriate range of angles when performing the exercise properly. If so, the exercise feedback module 280 may determine that the user is improperly performing the squatting exercise (i.e. bending towards to the floor during the squatting exercise).

Continuing with this example, in certain embodiments, the exercise feedback module 255 may be configured to generate one or more messages to provide feedback to the user about their posture and performance during the exercise. The one or more messages may then be transmitted to the user device 290 associated with the user from the computing device 210 via network 215. The user may then view messages provided by the exercise feedback module 255 on the user device 290. In certain embodiments, the user device 290 may receive the exercise feedback in terms of an instant audio feedback, stats/measurement review, and/or text messages from the computing device 210 via network 215. The user device 290 may be a suitable processor-driven device configured to facilitate the presentation of the one or more messages to the user. For example, the user device 290 may include one or more processors, memories, I/O interfaces, and/or network interfaces.

For example, if the exercise feedback module 255 determines that the user is bending towards the floor during the squatting exercise (i.e. the angle of the user's chest is outside the expected movement threshold), then one or messages may be generated that indicates the user is improperly bending towards the ground during the squatting exercise. The one or more messages may be transmitted from the computing device 210, which may be indicated as an instant audio feedback, such as an alarm or a buzz of the user device 290. On the user device 290, the user may receive reviews and text messages pertaining to the measurements and/or exercise information collected so that the user may correct his or her performance/posture accordingly.

As desired, embodiments of the disclosure may include a system 200 with more or less than the components illustrated in FIG. 2. Additionally, certain components of system 200 may be combined in various embodiments of the disclosure. The system 200 of FIG. 2 is provided by way of example only.

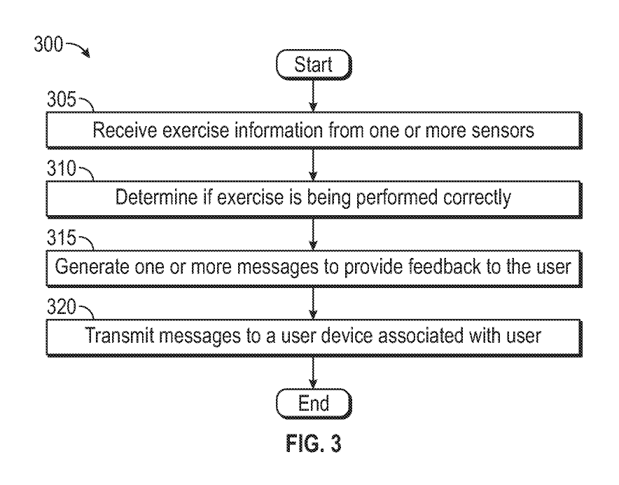

Referring now to FIG. 3, shown is a flow diagram of an example method 300 for improving fitness posture, according to an illustrative embodiment of the disclosure. The method 300 may be utilized in association with various systems, such as the system 200 illustrated in FIG. 2. In certain embodiments, the operations of the method 300 may be performed by a suitable computing device, such as the computing device 210 illustrated in FIG. 2.

The method 300 may begin at block 305. At block 305, exercise information associated with the movement of a user while performing an exercise may be collected and/or otherwise obtained. The exercise information may include values and measurements associated with the movement of a user while performing one or more exercises, such as a squat, which is collected by a plurality of sensors configured to monitor the motion of the user, such as, the sensors 150 illustrated in FIG. 1. As desired, the plurality of sensors may include a wide variety of different types of sensors, such as accelerometers and/or gyroscopic sensors. As described above, such exercise information associated with the user may be stored in a memory location, such as memory 225 in FIG. 2.

Next, at block 310, a determination as to whether the user is performing the exercise correctly based at least in part on the exercise information. For instance, the exercise feedback module, such as 255 in FIG. 2, may process the exercise information to determine, for example, the user is properly performing an exercise. In other instances, the exercise feedback module may determine that the user is using improper form or technique while performing the exercise. In certain embodiments, the exercise feedback module may make this determination by comparing the exercise data associated with the user to one or more pre-defined models that comprises information associated with the posture of a person correctly performing the exercise.

Thereafter, at block 315, based at least in part on the determination in block 310, one or more messages may be generated. The one or more messages may include various alerts, text, or visual aids to provide feedback to the user about improving the user's posture and performance of the exercise.

Following block 315, at block 320, the one or more messages may be transmitted to a user device, such as user device 290 in FIG. 2, associated with the user. For instance, the user device may be a mobile device associated with the user. In this way, the one or more messages may be provided to a user to monitor or improve their fitness posture and/or performance Preparation of the display results may include converting the one or more messages into a displayable format, creating or populating a user interface so the results can be displayed, transmitting the results to a display device, and/or rendering the results on a screen of a display device.

The method 300 of FIG. 3 may optionally end following block 320.

The operations described and shown in the method 300 of FIG. 3 may be carried out or performed in any suitable order as desired in various embodiments of the disclosure. Additionally, in certain embodiments, at least a portion of the operations may be carried out in parallel. Furthermore, in certain embodiments, less than or more than the operations described in FIG. 3 may be performed. As desired, the operations set forth in FIG. 3 may also be performed in a loop as a rotating machine is monitored.

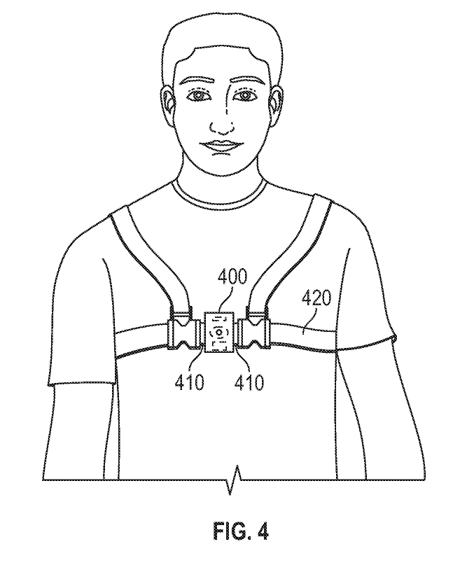

Referring now to FIG. 4, shown is a device for improving fitness posture as being worn by a user, according to an embodiment of the disclosure. As shown, the device 400 for improving fitness posture may be worn around the chest and/or torso of a user. In certain embodiments, the device 400 may comprise one or more attachment mechanisms 420 to attach the device 400 to one or more straps 420 that can be worn around the chest and/or torso of the user. Additionally, in certain embodiments, the device 400 may further include a leveling device to ensure stable mounting of the device 400 to the user.

The disclosure is described above with reference to block and flow diagrams of systems, methods, apparatus, and/or computer program products according to example embodiments of the disclosure. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some embodiments of the disclosure.

These computer-executable program instructions may be loaded onto a general purpose computer, a special purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, embodiments of the disclosure may provide for a computer program product, comprising a computer usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special purpose, hardware-based computer systems that perform the specified functions, elements or steps, or combinations of special purpose hardware and computer instructions.

While the disclosure has been described in connection with what is presently considered to be the most practical and various embodiments, it is to be understood that the disclosure is not to be limited to the disclosed embodiments, but on the contrary, is intended to cover various modifications and equivalent arrangements included within the spirit and scope of the appended claims.

This written description uses examples to disclose the disclosure, including the best mode, and also to enable any person skilled in the art to practice the disclosure, including making and using any devices or systems and performing any incorporated methods. The patentable scope of the disclosure is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The claimed invention is:

1. A system for improving fitness posture, comprising:
one or more sensors configured to collect exercise information associated with the movement of a user while the user is performing an exercise; and
at least one processor configured to access at least one memory and execute computer-executable instructions to:
receive the exercise information from the one or more sensors;
determine if the user is performing the exercise correctly based at least in part on the exercise information;
generate, based at least in part on the determination, one or more messages to provide feedback to the user about improving the user's posture and performance of the exercise; and
transmit the one or more messages to a user device associated with the user.

2. The system of claim 1, wherein the one or more sensors comprise one or more accelerometers and gyroscopes.

3. The system of claim 1, wherein the one or more sensors are attached to the torso or upper body of the user.

4. The system of claim 1, wherein the exercise information comprises forward and backward movements, side-to-side movements, and upward and downward movements of the user while the user is performing the exercise.

5. The system of claim 1, wherein the exercise information comprises measurements and values associated with the movement and rotation of the user's body relative to a starting position of the user.

6. The system of claim 1, wherein the exercise the user is performing is a squat.

7. The system of claim 1, wherein the computer-executable instructions to determine if the user is performing the exercise correctly is based at least in part on a comparison between the exercise information associated with the user and one or more pre-defined models.

8. The system of claim 1, wherein the computer-executable instructions to determine if the user is performing the exercise correctly is based at least in part on a comparison between the exercise information associated with the user and training data associated with the posture of a person correctly performing the exercise.

9. The system of claim 1, wherein user device is a mobile device.

10. A device for improving fitness posture, comprising:
a housing unit configured to attach to the body of a user, wherein the housing unit comprises:
at least one aperture;
a laser disposed within the housing unit and behind the at least one aperture, wherein the laser is configured to project a light through the at least one aperture and onto a surface to aid the user while performing one or more exercises;
one or more sensors disposed within the housing unit that are configured to collect exercise information associated with the movement of the user while performing one or more exercises; and
at least one processor disposed within the housing unit, wherein the at least one processor is in communication with the one or more sensors, and wherein the at least one processor is configured to:
receive the exercise information from the one or more sensors;
determine if the user is performing the one or more exercises correctly based at least in part on the exercise information;
generate one or more messages to provide feedback to the user about improving the user's posture and performance of the one or more exercises; and
transmit the one or more messages to a user device associated with the user.

11. The device of claim 10, wherein the housing unit further comprises one or more attachment mechanisms to attach the housing unit to one or more straps.

12. The device of claim 11, wherein the one or more straps are configured to be worn around the chest or torso of the user.

13. The device of claim 10, wherein the housing unit may further comprise a leveling device to ensure stable mounting of the housing unit to the body of the user.

14. The device of claim 10, wherein the one or more sensors comprise one or more accelerometers and gyroscopes.

15. The device of claim 10, wherein the exercise information comprises forward and backward movements, side-to-side movements, and upward and downward movements of the user while the user is performing the one or more exercises.

16. The device of claim 10, wherein the exercise information comprises measurements and values associated with the movement and rotation of the user's body relative to a starting position of the user.

17. The device of claim 10, wherein the one or more exercises is a squatting exercise.

18. A method for improving fitness posture, the method comprising:
receiving exercise information from the one or more sensors, devices, or computing devises, wherein the exercise information comprises one or more values or measurements associated with the movement or rotation of a user while the user is performing an exercise;
determining if the user is performing the exercise correctly based at least in part on the exercise information;
generating, based at least in part on the determination, one or more messages to provide feedback to the user about improving the user's posture and performance of the exercise; and
transmitting the one or more messages to a user device associated with the user.

19. The method of claim 18, wherein the one or more sensors comprise one or more accelerometers and gyroscopes.

20. The method of claim 18, wherein the one or more sensors are attached to the user's torso or upper body during the performance of the exercise.
